(19) United States
(12) Patent Application Publication
(10) Pub. No.: US 2004/0064072 A1
(43) Pub. Date: Apr. 1, 2004

(54) System and method for monitoring changes in body position

(76) Inventor: Shmuel Shapira, Sherwood, OR (US)

Correspondence Address: KOLISCH HARTWELL, P.C., 520 S.W. YAMHILL STREET, SUITE 200, PORTLAND, OR, 97204, US

(21) Appl. No.: 10/665,890

(22) Filed: Sep. 19, 2003

Related U.S. Application Data
(60) Provisional application No. 60/415,112, filed on Sep. 30, 2002.

(51) Int. Cl. A61B 5/103

(52) U.S. Cl. 600/595

(57) ABSTRACT

A system and method for monitoring the position of an individual. According to one aspect of the disclosure, one or more position detectors are associated with an individual, so that movement of the individual results in a corresponding movement of the position detector(s). The position detector(s) are monitored to identify such movements. The movements of an individual may be tracked, so that notification may be provided if an individual remains stationary for longer than a predetermined period of time.

### CROSS REFERENCE TO RELATED APPLICATIONS

This application claims the benefit of U.S. Provisional Patent Application No. 60/415,112, filed Sep. 30, 2002. The content of the above referenced application is herein incorporated by reference for all purposes.

### BACKGROUND

Many conditions or circumstances may lead to an individual resting in the same position for extended periods of time. For example, an individual may lie in bed without the capacity to change position due to a health condition and/or old age. Lying in the same position for too long may result in undesirable health consequences, such as bedsores.

### SUMMARY

A system and method for monitoring the position of an individual is provided. According to one aspect of the disclosure, one or more position detectors are associated with an individual, so that movement of the individual results in a corresponding movement of the position detector(s). The position detector(s) are monitored to identify such movements. The movements of an individual may be tracked, so that notification may be provided if an individual remains stationary for longer than a predetermined period of time.

### BRIEF DESCRIPTION OF THE DRAWINGS

FIG. 1 is a schematic view of a monitoring system configured to monitor a position of an individual.

FIG. 2 is a schematic view of the individual of FIG. 1 after the individual has significantly moved from the position of FIG. 1.

### DETAILED DESCRIPTION

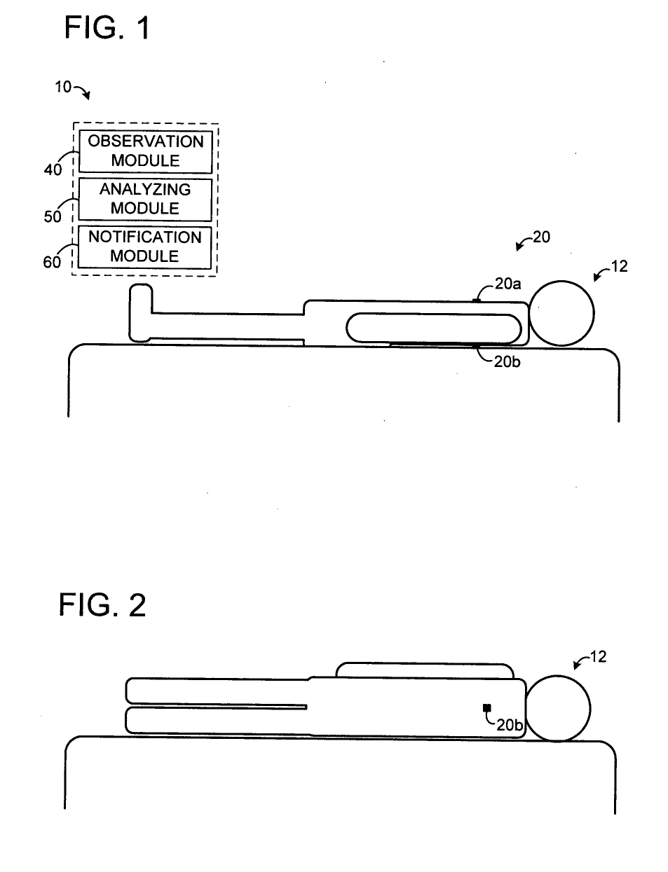

FIG. 1 schematically shows a position monitoring system 10, which may be used to monitor the position of an individual 12. Monitoring system 10 may be used to monitor the time that individual 12 remains in the same position. Such monitoring may be helpful in controlling the occurrence of bedsores, which may be caused by prolonged pressure on an area of the skin that is in contact with a bed. If an individual remains in substantially the same position for longer than a predetermined period of time, the monitoring system can provide notification, so that the individual may be turned, or otherwise assisted in avoiding bedsores or other negative consequences associated with remaining in the same position for too long.

In an exemplary embodiment, monitoring system 10 may include one or more position detectors 20, which may be attached to individual 12 so that any significant change in body position of the individual will cause a measurable movement of at least one detector. For example, as shown in FIG. 2, if the individual turns in bed, one or more position detectors will move with the individual, thus resulting in a measurable movement of the position detector(s). In general, a significant shift in position is one that correlates with a different area of the body being in contact with a bed. Detection of accurate direction and/or exact amount of movement may not be necessary to adequately identify if an individual has been stationary for longer than a desired amount of time. A position detector may be attached to the person's body, garment, absorbent article, etc. Position detectors may be attached, or otherwise associated with, at least one of a head, shoulder, arm, hand, chest, back, hip, pelvic region, buttocks, leg, foot, or other body part. In the illustrated embodiment, a position detector 20a services a chest region and a position detector 20b services a back region.

Monitoring system 10 may include an observation module 40 configured to monitor the position of the position detectors. Observation module 40 may be configured to continuously or intermittently monitor the position detectors. Furthermore, the observation module and position detectors may be collectively configured to assess the relative position of the detectors in some embodiments, and the absolute position of the detectors in some embodiments. Observation module 40, as well as other elements of monitoring system 10, has been schematically represented. The size, shape, and appearance of the observation module, may be chosen according to a desired application and/or usage environment.

The monitoring system may include an analyzing module 50, configured to utilize information acquired via the observation module to identify when an individual has been stationary for longer than a desired time. For example, analyzing module 50 may utilize the relative and/or absolute position of the position detectors, as observed by observation module 40, to determine a corresponding position of individual 12. In this manner, monitoring system 10 may be used to determine if the individual remains in the same position for longer than a predetermined period of time. Such a period of time may be determined to minimize the negative affects of bedsores or other harmful conditions. The analyzing module may include a system controller for performing a variety of supervisory functions including data acquisition and storage, decision-making, scheduling, coordination, and execution of the other various system functions. The system controller may include a processor, such as an embedded hardware microcontroller, which may include, or interface with, data storage devices and/or peripheral devices, such as timers, counters, I/O ports, etc. In some embodiments, analyzing module 40 may take the form of a multipurpose computer executing software configured to utilize information acquired via the observation module to identify when an individual has been stationary for longer than a desired time. In such embodiments, the observation module may be a peripheral configured to operatively interact with the multipurpose computer.

Monitoring system 10 may include a notification module 60 configured to convey information pertaining to the position of individual 12. For example, a notification module may be configured to convey the length of time a person has remained in one position, that a pre-set time has elapsed without a change in position, the frequency of position changes, and/or other information. The notification module may include audio and/or visual indicators for conveying information regarding the position of an individual, and/or other information. In some embodiments, data regarding the position of an individual may be transferred to another system via a wired or wireless communication interface.

In some embodiments, portions of a monitoring system may be integrated with a bed and/or other device. In some embodiments, the observation module, analyzing module, and/or notification module may be housed in a common case, and in some embodiments the monitoring system may be comprised of several separated elements operatively coupled to one another, such as via electrical or wireless information links. As mentioned above, at least some of the functionality of the monitoring system may be executed on a multipurpose computer, such as a personal computer.

Monitoring system 10 may be variously configured to assess the position of an individual. In some embodiments, the monitoring system may utilize position detectors that include pressure sensors, temperature sensors, or other devices which may be used to determine the position of an individual.

The following description focuses on nonlimiting examples of exemplary position detectors in the form of remote energy sensors. The remote energy sensors are configured to absorb, reflect, radiate, and/or propagate energy supplied by a remote source, such as an energy field delivered via an observation module of the monitoring system. Such delivered energy may be magnetic, electromagnetic, optical, mechanical, or other. Energy sensors in the form of transponders similar to those used by Radio Frequency Identification (RFID) and Electronic Article Surveillance (EAS) systems may be used in some embodiments. An energy sensor may be energized, at least partially, through contact-less coupling, and the exchanged energy between the energy sensor and other parts of the system may be measured and analyzed to identify movement in space of the energy sensor.

An observation module may include an energizing module that is capable of energizing an energy sensor periodically or continuously. Consecutive energizing may vary in properties such as a change in frequency or amplitude. More than one energizing module may be employed to facilitate differential measurement of exchanged energy.

An analyzing module may monitor properties of energy that is absorbed, reflected, radiated, and/or propagated by the energy sensor. The observation module may repeatedly measure at least one property of the energy, such as its amplitude, and supply the analyzing module with information regarding the measurement. The analyzing module may identify a variation between consecutive measurements that is indicative of a significant shift in the position of the energy sensor relative to the observation module. A significant shift in an energy sensor position may be considered one that correlates with a change in the monitored person's pose wherein a different area of the body is in contact with the bed.

In some embodiments, an energy sensor may include a resonator such as an inductive/capacitive resonating circuit. An observation module may include one or more exciting coils through which an alternating magnetic field in one or more frequencies can be induced. Changing current in an exciting coil may vary the magnetic flux through the resonator, and thus, may induce an electromotive force in the resonator. Therefore, the observation module and the energy sensor may engage in mutual inductance with one another, as well as other participating elements, such as other energy sensors or the analyzing module. When certain conditions prevail, the energy sensor will enter a state of resonance, which is characterized by measurable increase in energy transferred to the resonating circuit.

The system may utilize magnetic energy and magnetic coupling to power one or more energy sensors and track changes in the interaction among system components. In general, the quantum of energy that is exchanged between an exciting coil and an energy sensor depends on the magnetic coupling coefficient, their relative impedance, and the frequency of the oscillating magnetic field. Mapping the behavior of the system at different frequencies generates a response curve representing the energy transfer pattern. A significant movement of an energy sensor can affect the coupling coefficient and generate a system response curve that may be distinctly different from one observed before the shift. Therefore, response curves acquired from different measurements may be analyzed to determine an individual's position, or at least if an individual's position has changed.

A data acquisition cycle may involve a frequency sweep within a predetermined range while concurrently sampling (measuring) a signal representing the level of energy in a chosen point in the system. Signal acquisition may be achieved by means of analog to digital conversion technique, whereby the measured signal is successively sampled at fixed intervals to produce a series of discrete measurement values that, in turn, are stored in system memory. The signal-sampling rate may be synchronized with the rate of change in frequency (sweep rate) generated by the observation module. The net result of the acquisition cycle may be an array of numerical values, stored in memory, representing the instantaneous energy for each frequency quantum in the sweep range. Using the information stored in this array allows the depiction of the acquired signal frequency-amplitude response curve.

A data analysis process may involve comparing measured results and/or calculated response curves to those derived from a previous data acquisition cycle. A change in an energy transfer pattern that is above a certain allowed tolerance may indicate a change in the position of the monitored individual. When two or more exciting coils are utilized, a data acquisition cycle may be performed for each coil and the results associated with each coil may be compared to the results from a previous cycle of the same coil. A plurality of exciting coils may provide comparative information that may assist in detection of movement or validate results.

An environment in which an individual is located may present background noise and/or other types of interference that may affect measured energy distributions. To minimize the negative affects of such interference, a baseline interference may be calculated, and operatively subtracted from measurements taken while monitoring an individual's position. Some types of interference anomalies may be identified, and effectively ignored during monitoring, so that such anomalies do not result in false determinations that an individual has moved.

In some embodiments, a position detector may be configured with characteristics that differentiate it from other detectors. As a nonlimiting example, a position detector may be configured to modify energy exchange to include data, such as an identifier, or any other unique property that will differentiate it from other detectors. In some embodiments, different detectors may respond differently to certain frequencies, or may apply unique modulation to exchanged energy. One or more distinguishable detectors may be configured to be shielded by the body in certain postures, therefore enabling detection of movement by a change in the conveyed data received by the observation module. In addition, the use of identification data may provide the possibility to utilize a single observation module and/or analyzing module for more than one person.

Although the present disclosure has been provided with reference to the foregoing operational principles and embodiments, it will be apparent to those skilled in the art that various changes in form and detail may be made without departing from the spirit and scope defined in the appended claims. The present disclosure is intended to embrace all such alternatives, modifications and variances. Where the disclosure or claims recite “a,” “a first,” or “another” element, or the equivalent thereof, they should be interpreted to include one or more such elements, neither requiring nor excluding two or more such elements.

What is claimed is:

1. A method for monitoring body-position changes of an individual, comprising:
attaching a position detector to the individual;
energizing the position detector; and
conducting a plurality of measurements to identify a change in a characteristic of energy associated with the position detector, wherein such a change is indicative of significant movement of the position detector and a corresponding change in position of the individual.

2. The method of claim 1, wherein the position detector is one of a plurality of position detectors.

3. The method of claim 2, wherein at least one of the position detectors is distinguishable from at least one other of the position detectors.

4. The method of claim 1, wherein the position detector is a transponder.

5. The method of claim 1, further comprising tracking time since the individual has moved.

6. The method of claim 5, further comprising reporting that the individual should be moved if a predetermined period of time has passed since the individual has moved.

7. A system for monitoring body position changes of an individual, comprising:
a position detector;
an observation module configured to measure energy associated with the position detector; and
an analyzing module configured to identify a change in a characteristic of energy associated with the position detector by comparing a plurality of measurements taken by the observation module, wherein such a change is indicative of significant movement of the position detector and a corresponding change in position of the individual.

8. The system of claim 7, wherein the analyzing module includes a timer configured to track how long the individual remains stationary.

9. The system of claim 7, further comprising a notification module for conveying that the individual has not moved for a predetermined period of time.

10. The system of claim 7, wherein the position detector is one of a plurality of position detectors.

11. The system of claim 10, wherein at least one of the position detectors is distinguishable from at least one other of the position detectors.
